(19) United States
(12) Patent Application Publication
(10) Pub. No.: US 2012/0322144 A1
(43) Pub. Date: Dec. 20, 2012

(54) MICROFLUIDIC DEVICES AND SYSTEMS

(75) Inventors: Wilbur A. Lam, Decatur, GA (US); David Myers, Atlanta, GA (US); Zhengchun Peng, Beaverton, OR (US); Gang Bao, Mableton, GA (US)

(73) Assignee: EMORY UNIVERSITY, Atlanta, GA (US)

(21) Appl. No.: 13/523,203

(22) Filed: Jun. 14, 2012

Related U.S. Application Data
(60) Provisional application No. 61/496,799, filed on Jun. 14, 2011.

(51) Int. Cl. C12M 1/26

(52) U.S. Cl. 435/309.1

(57) ABSTRACT

A device may be configured to allow manipulation of the cell(s), such as test or drug applications, independent of the operation and/or structure of the device. The device may be configured to separate, hold and release at least one cell suspended in a fluid. The device may include a region configured to receive the fluid including at least one cell; a cell holding device that is in open communication with the region and includes at least one cell isolation region configured to releasably separate and hold each cell; and a passage that is configured to receive and supply pressure to the cell holding device. The device may further include a second region that includes the cell holding device and is in fluid communication with the region.

### CROSS-REFERENCE TO RELATED APPLICATION

This application is a non-provisional of Application Ser. No. 61/496,799, filed Jun. 14, 2011, which is hereby incorporated by this reference in its entirety.

### GOVERNMENT ACKNOWLEDGMENT

This invention was made with government support under Grant 2PN2EY018244-06 awarded by the National Institutes of Health. The government has certain rights in the invention.

### BACKGROUND

Cell-based therapy, such as gene correction, represents a powerful new paradigm in which genetic diseases and cancer may be cured by correcting the causative mutations in the genetic code. Several techniques currently exist for gene correction either by using biological methods techniques such as viral vectors or physically injecting therapeutic agents into cells. Viral methods require time consuming optimization that is difficult to predict and off-target effects may alter other parts of the genome, potentially leading to cancerous mutations. Physical methods such as electroporation have low efficiency and may not deliver the target therapeutic to cell nuclei.

There is a need for a device capable of separating, holding, and releasing cells in a high-throughput manner.

### SUMMARY

The disclosure relates to a microfluidic device configured to separate, hold and release at last one cell suspended in a fluid in a high-throughput manner. The microfluidic device may be configured to include multilayers. The microfluidic device may be configured to hold at least one cell that is intended to receive material from a delivery device. The microfluidic device may be configured to be used in conjunction with a delivery device, such as a microneedle.

In some embodiments, the disclosure relates to a device configured to separate, hold and release at least one cell suspended in a fluid. The device may comprise a region configured to receive the fluid including at least one cell; a cell holding device that is in open, fluid communication with the region and includes at least one cell isolation region configured to separate, hold and release a cell; and a passage that is configured to receive and supply pressure to the cell holding device.

In some embodiments, the cell isolation region may include an opening that is configured to support a surface of the cell. The region may be disposed substantially at a depth that is same as the cell holding device. The device may further comprise a second region that includes the cell holding device and is in fluid communication with the region. The region may be disposed above the second region.

In some embodiments, the cell holding device may be disposed above the passage. In some embodiments, the cell holding device may include an array of a plurality of cell isolation regions. In some embodiments, each of the plurality of cell isolation regions may be of a substantially a same size. In other embodiments, the plurality of cell isolation regions may be of different sizes. In some embodiments, the device may further comprise a support substrate, wherein the region, cell holding device and passage are disposed above the support substrate.

In other embodiments, the device may include a cell receiving region configured to receive the fluid including at least one cell; a collection region that is in fluid communication with the cell receiving region, the collection region including a cell holding device configured to separate and hold each cell; and a passage that is disposed adjacent to the cell collection region. The passage may be disposed below the cell receiving region and the cell holding device. The collection region may have a middle section, a first end and a second end, wherein the collection region has a width that tapers from the middle section towards each end. The cell holding device may be disposed within the middle section of the collection region.

In further embodiments, the device may further comprise an outlet passage having one end disposed on a side of the collection region and a second end near or close to a side of the device. The outlet passage may be configured to allow the fluid to exit the collection region. The outlet passage may also be configured to prevent the at least one cell to be isolated to exit the collection region. The outlet passage may be configured to receive and supply pressure.

In other embodiments, the disclosure relates to a system configured to separate, hold and release at least one cell suspending in a fluid. The system may include a microfluidic device. The microfluidic device may include a region configured to receive the fluid including at least one cell; a cell holding device configured to separate, hold and release each cell; and an outlet passage having one end disposed on a side of the collection region and a second end near or close to a side of the cell isolating device and configured to control movement of the at least one cell within the microfluidic device. The system may further comprise a pressure source configured to connect to the outlet passage to supply at least one of a positive pressure or a negative pressure. In some embodiments, the system may further comprise at least one delivery device configured to deliver a material to a separated cell.

### DESCRIPTION OF FIGURES

The disclosure can be better understood with the reference to the following drawings and description. The components in the figures are not necessarily to scale, emphasis being placed upon illustrating the principles of the disclosure.

FIG. 1 shows a microfluidic device according to some embodiments;

FIG. 2 shows a partial enlarged view of the device of FIG. 1;

FIG. 3 shows another partial enlarged view of the device of FIG. 1;

FIG. 4 shows a cross-section of the device of FIG. 1;

FIG. 5 shows an example of a cell holding device according to some embodiments;

FIG. 6 shows another example of a cell holding device according to some embodiments;

FIG. 7 shows another example of a cell holding device according to some embodiments;

FIG. 8 shows an operational example of the microfluidic device;

FIG. 9 shows another operational example of the microfluidic device; and

FIG. 10 shows an enlarged view of a delivery device.

### DESCRIPTION OF EMBODIMENTS

The following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

As used herein, “hold,” as it related to at least one cell relative to a surface, means to support or hold the cell(s) relative to a surface. In some embodiments, the cells are held above a surface. In further embodiments, the cells are held at least partially within an opening of the surface.

Any direction referred to herein, such as “top,” “bottom,” “left,” “right,” upper,” “lower,” “above,” “below,” and other directions and orientations relative to the microfluidic devices are described herein for clarity in reference to the figures are not limiting of an actual device or system or use of the device or system. Devices or systems as described herein may be used in a number of directions or orientations.

In various embodiments, the microfluidic devices may be configured to allow controlled mechanical methods to individually hold and separate at least one cell for test or drug applications. The microfluidic devices may be configured to controllably separate, hold, and release cell(s).

According to embodiments, because the devices are configured to controllably separate, hold and release a cell, the delivery of material to a cell, such as to a cell nucleus, may be improved. The biochemistry may be possibly decoupled from the delivery method, enabling optimization of the delivery, such as each component of gene therapy, independently.

In some embodiments, the microfluidic devices may include at least one region that is in fluid communication with an outlet passage or channel. The at least one region may be configured to receive at least cell suspended in a fluid (hereinafter “fluid suspension”). The at least one (or a first) region or an additional (or a second) region may further include a holding device that is configured to (controllably) separate, hold and release at least one cell suspended in a fluid (hereinafter “fluid suspension”) substantially from that fluid suspension. In some embodiments, the outlet channel or passage may be configured to allow a portion of the suspension fluid to exit a region, and/or receive and/or supply a pressure (positive or negative), such as a holding or a releasing pressure. The at least one region and outlet channel or passage may be configured to be in open communication, for example, at least one region or channel may be exposed (e.g., not covered by a top surface). Thus, the microfluidic devices may be configured to allow manipulation of the cell(s), such as test or drug applications, independent of the operation and/or structure of the microfluidic device.

It will be understood that microfluidic devices and systems according to embodiments may be configured to be receive and individually isolate or separate any type(s) of cells from a fluid suspension. These cells may be any type of biological cells as well as mammalian cells, etc. For example, the cells may include but are not limited to stem cells, such as hemapoitec, blood cells, Eukaryotic cells, epithelial, lung cells, embryonic cells, fetal cells, tumor cells, and infectious organism (e.g., bacteria, protozoa, and fungi). The cells may be of any amount. Although the disclosure refers to “cells,” it will be understood that “cells” may be one (single) cell of a type, more than cell of a type, or more than cell of a combination of different types and amounts. The cell(s) may be further included in a fluid suspension. The cells may also be included in any known fluid suspension, such as blood.

It will be further understood that the microfluidic devices and systems according to embodiments may be configured or adjusted according to type or property of cell(s) (e.g., size, shape, deformability, and surface characteristics), and number of cells to be separated. For example, the dimensions of the microfluidic devices as well as the specific dimensions of each region and passage (or channel) may be adjusted. The microfluidic devices and systems may be further configured or adjusted according to the fluid suspension used. For example, the devices and systems may be configured or adjusted according to the characteristics of the fluid suspension, such as the viscosity and amount.

In some embodiments, the devices may be sterilized. In further embodiments, the devices may be a single, use device. In further embodiments, the devices may be disposable.

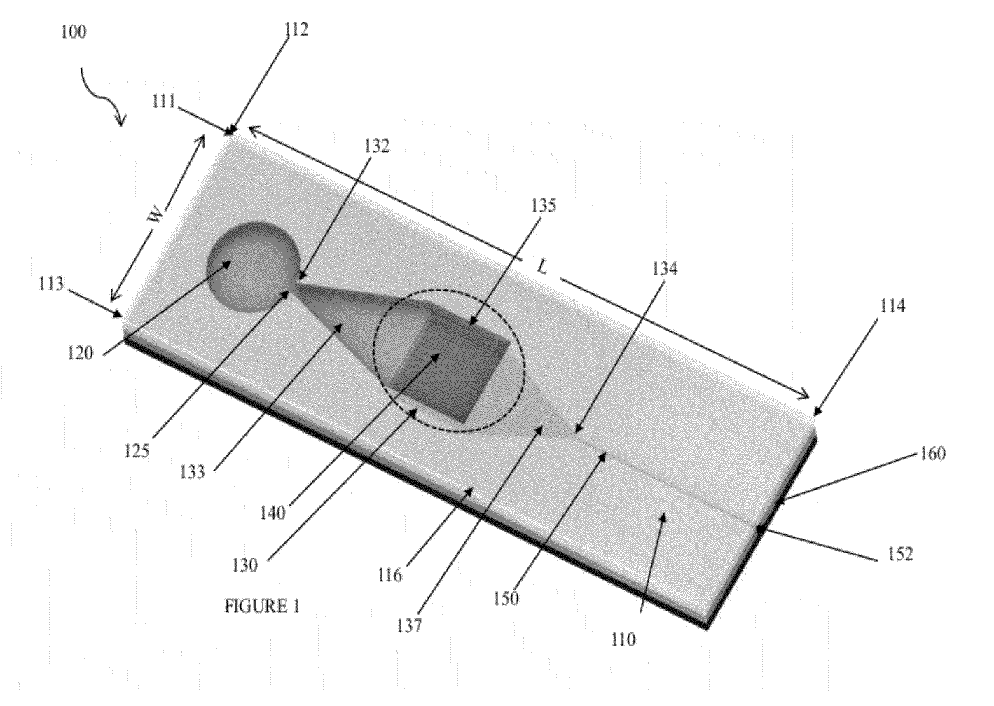

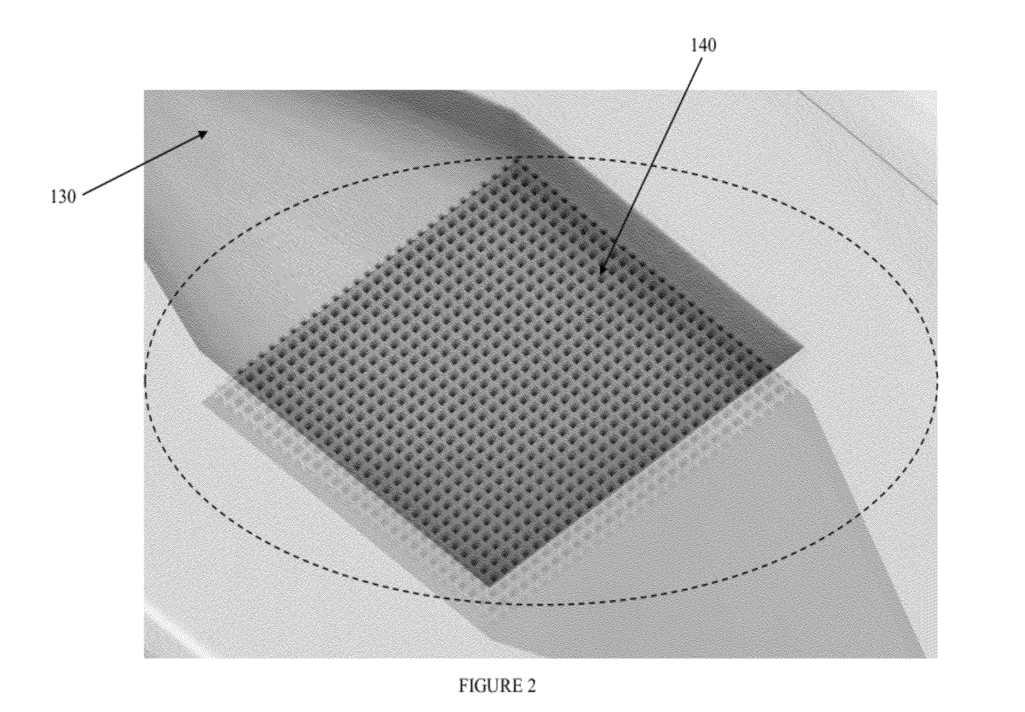

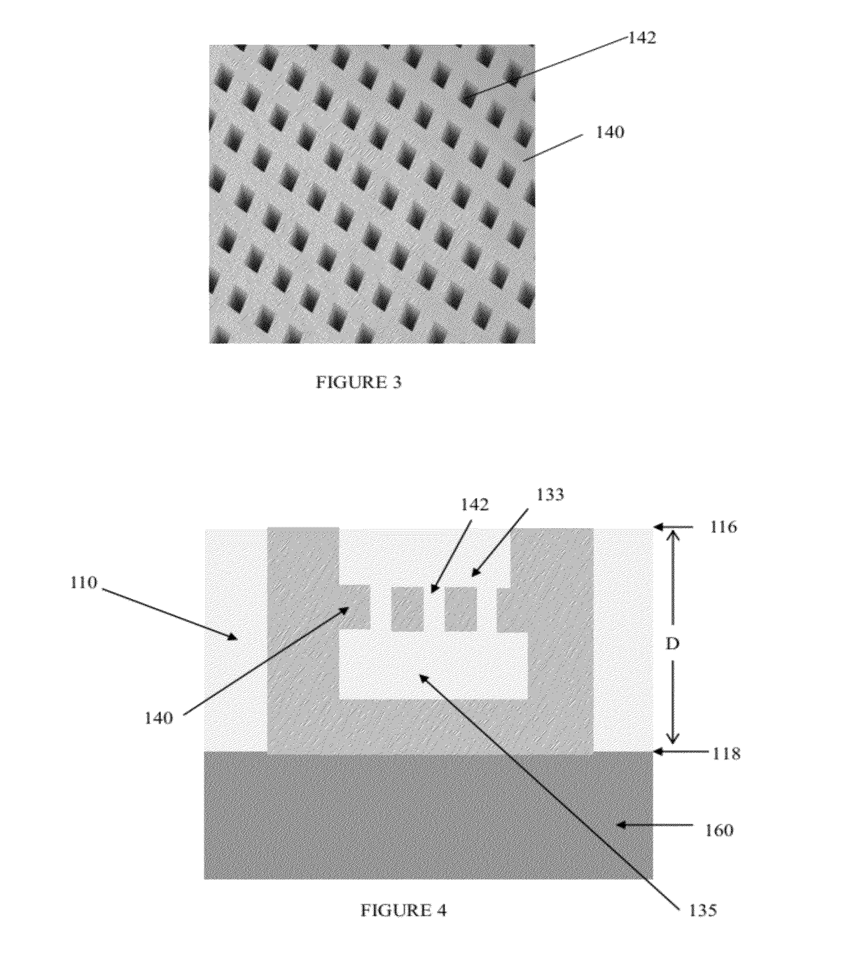

As shown in FIGS. 1 through 4, a microfluidic device 100 may include a cell isolation body 110. In some embodiments, the cell isolation body 110 may have a rectangular shape. In other embodiments, the cell isolation body 110 may have a different shape. The cell isolation body 110 may have any shape. In some embodiments, the shape of the cell isolation body 110 may depend on system requirements.

In some embodiments, the cell isolation body 110 may have a (first) side 112 and an opposing (second) side 114. The cell isolation body 110 may have a length (L) between the first side 112 and the second side 114. The cell isolation body may have a (third) side 111 and an opposing (fourth) side 113. The cell isolation body may have a width (W) (transverse to the length) between the third side 111 and the fourth side 113. The cell isolation body 110 may have a (first) surface 116 and an opposing (second) surface 118. The cell isolation body 110 may have a depth (D) between the first surface 116 and the second surface 118. The cell isolation body 110 is not limited to the length, width, and depth shown in the figures. The cell isolation body 110 may have any length, width, or depth. In some embodiments, the cell isolation body 110 may include at least one region configured to receive the cells for individual separation or isolation from a suspended fluid.

As shown in FIG. 1, in some embodiments, the microfluidic device 100 may include a cell receiving region 120. The cell receiving region 120 may be configured to receive the cells for individual separation. In further embodiments, the cell receiving region 120 may also be configured to receive the released cells, which are the cells released from separation.

In some embodiments, the cell receiving region 120 may be a cavity disposed within the cell isolation body 110. The cell receiving region 120 may be of any shape and size. In some embodiments, the cell receiving region 120 may have a circular shape. However, the cell receiving region 120 is not limited to the circular shape as shown in the figures. In other embodiments, the cell receiving region 120 may have a square, rectangular, triangular, a trapezoidal, or an asymmetrical shape, as well as any symmetrical shape.

In some embodiments, the cell receiving region 120 may have one depth. In other embodiments, the cell receiving region 120 may have more than one depth, such as a gradual decrease or increase in depth. For example, the depth of the cell receiving region 120 may taper towards an end, both ends, as well as towards the middle. The cell receiving region 120 may also have any width (e.g., diameter) or length.

In some embodiments, the cell isolation body 110 may further include a region configured to individually separate at least one cell substantially from a suspended fluid. In some embodiments, the region may be a separate region (also referred to as a “second region”) from the cell receiving region 120. In some embodiments, the region may be configured to receive cells from the cell receiving region 120. This region may be configured to also collect the released cell(s) released from the separation in addition to or in the alternative of the cell receiving region 120.

As shown in FIGS. 1 through 4, the cell isolation body 110 may include a (cell) collection region 130. The collection region 130 may be configured to individually separate each cell and releasably hold each cell.

The collection region 130 may have any shape (with respect to the top plan view or transverse cross-sections). The collection region 130 may have a (first) end 132 and an opposing (second) end 134, and a length therebetween. In some embodiments, the collection region 130 may have a symmetrical shape along its length.

In some embodiments, the collection region 130 may include a plurality of sections. In some embodiments, the collection region 130 may include at least one section that has a width that tapers towards at least one end. As shown in FIG. 1, the collection region 130 may include at least two sections 133 and 137 that have tapered widths. In other embodiments, the collection region 130 may include more or less tapered sections. In some embodiments, the sections 133 and 137 may be disposed on either side of a middle section 135.

In some embodiments, the width of the at least one of the sections 133 and 137 may taper or narrow from the middle section 135 toward each or both ends. In other embodiments, the width of the at least one of the sections 133 and 137 may increase from each end toward the middle section 135.

In some embodiments, the sections 133 and 137 may have the substantially the same shape. In some embodiments, the sections 133 and 137 may have a triangular shape. In other embodiments, the sections 133 and 137 may have a different shape. In some embodiments, the section 135 may have a rectangular or square shape. In other embodiments, the section 135 may have a different shape.

In other embodiments, the collection region 130 may have a uniform shape along its length. In further embodiments, the collection region 130 may have an asymmetrical shape. The collection region 130 may also have any width (or diameter), depth, or length.

In some embodiments, the collection region 130 may have one or the same depth and/or may be disposed at the same depth along its length. In some embodiments, at least one of the sections of the collection region 130 may have and/or may be disposed at substantially the same depth as the cell receiving region 120. In some embodiments, at least one of the sections of the collection region 130 may have and/or may be disposed at a depth that is different from the cell receiving region 120. In some embodiments, the depth of at least one section of the collection region 130 may be larger than the depth of the cell receiving region.

In other embodiments, the collection region 130 may have more than one depth along its length. In some embodiments, the collection region 130 may have a depth that tapers, e.g., gradually increases or decreases along its length. In some embodiments, the depth of the collection region 130 may taper towards an end, towards both ends, or towards the middle. In some embodiments, the collection 130 region may have a depth that gradually increases from the end closest to the cell receiving region 120 to the opposite end.

In some embodiments, at least one section of the collection region 130 may have a different depth. In some embodiments, at least one depth may be substantially same depth as the cell receiving region 120. In other embodiments, at least one depth of the collection region 130 may be greater than the cell receiving region 120. In alternative embodiments, at least one depth of the collection region 130 may be smaller (i.e., has a lesser height) than the cell receiving region 120.

In some embodiments, at least one section of the collection region 130 may be disposed at different depths. The sections 133, 135, and 137 of the collection region 130 may be disposed at different depths of the cell isolation body 110. In some embodiments, at least one of the sections of the collection region 130 may be disposed at the same depth as the cell receiving region 120. In some embodiments, the section 133 may be disposed at the same depth as the cell receiving region 120. In some embodiments, at least one section of the collection region may be disposed at a depth lower than the cell receiving region 120. In some embodiments, the sections 135 and 137 may be disposed at a depth lower than the cell receiving region 120. In some embodiments, at least two of sections of the collection region 130 may be disposed at the same depth.

In some embodiments, the collection region 130 may have the same or different volume capacity as the cell receiving region 120. Notwithstanding the shape, in some embodiments, the collection region 130 may have a greater volume than cell receiving region 120.

In some embodiments, the collection region 130 may be in fluid communication with the cell receiving region 120. In some embodiments, the collection region 130 may be in fluid communication with the cell receiving region 120 via at least one (first) fluid communication passage. The fluid communication passage may be disposed at any point or location between the cell receiving region 120 and the collection region 130, as well as at any depth. There may also may more than one fluid communication passage. The fluid communication passage may be an opening, a channel or fluid conduit of any length, depth, width and shape. The fluid communication passage may include but is not limited to a microfluidic channel. In some embodiments, the fluid communication passage may have a width that is the narrower or smaller than the collection region 130 and/or cell receiving region 120.

As shown in FIG. 1, the device 110 may include a passage 125 between the collection region 130 and cell receiving region 120. As shown in FIG. 1, the passage 125 may be disposed at the end 132 of the collection region 130 having the narrowest width. In some embodiments, the passage 125 may be disposed at and/or may have the same depth as the cell receiving region 120 and at least one section of the collection region 130. In some embodiments, the passage 125 may have the same depth and be disposed at the same depth as the cell receiving region 120 and at least section 133.

In other embodiments, the passage 125 may have a greater depth than the cell receiving region 120 but a lesser depth than the collection region 130. In further embodiments, the passage 125 may have a tapered depth that bridges the depths of the cell receiving region 120 and the collection region 130.

The cell isolation body 110 may further include at least one cell holding device configured to individually and releasably separate and hold at least one cell cell(s). The cell holding device may be disposed within any region of the cell isolation body 110. The cell holding device may be disposed within the collection region 130, the cell receiving region 120, or another region, such as additional regions.

In some embodiments, the cell holding device may be configured to controllably separate some, most, or all cells provided in the fluid suspension and individually hold each separated (or isolated) cell. The cell holding device may be configured to separate any number of cells, including on cell, substantially from the fluid suspension provided and individually hold each separated cell. The cell holding device may include at least one cell isolation region, each region being configured to individually hold a cell. The cell isolation region may be configured to separate at least one cell when the fluid flows across the cell holding device. A cell may be considered to be separated from the fluid suspension when the cell is positioned or disposed within or above a cell isolation region, for example, as shown in FIGS. 8 and 9. In some embodiments, a portion of a surface of the cell may contact the cell isolation region when being held by the cell device.

As shown in FIG. 1, the cell isolation body 110 may include a cell holding device 140. The cell holding device 140 may be disposed within the cell collection region 130. The cell holding device 140 may be disposed at one of the sections of the cell collection region 130. In some embodiments, the cell holding device 140 may be disposed at the widest section of the collection region 130, e.g., the section 135. In other embodiments, the cell holding device 140 may be disposed at other locations and/or regions.

Although FIG. 1 shows one cell holding device 140, in other embodiments, the cell isolation body 110 may include more than one cell holding device. Each holding device(s) may be substantially identical or different. For example, each holding device may be configured for holding a different cell.

The cell holding device 140 may be a planar body with at least cell isolation region 142. The cell holding device 140 may include any number of cell isolation regions 142. However, the at least one cell isolation region 142 may be of any shape and planar relationship. In some embodiments, the cell isolation region(s) 142 may have a symmetrical shape, asymmetrical shape, or a combination thereof. The shape may include but is not limited to circular, square, or trapezoidal shape.

Also, in some embodiments, the cell isolation region 142 may be disposed within and/or disposed on a surface of the cell holding device 140. In some embodiments, as shown in the figures, the cell isolation region 142 may include an opening. In some embodiments, the cell isolation region 142 may additionally and/or optionally include a depression, a protrusion, or combination thereof. In some embodiments, the cell isolation region 142 may include a protrusion that completely surrounds or partially surrounds an opening and/or depression.

In some embodiments, there may be one cell isolation region 142. In other embodiments, there may be more than one cell isolation region 142. There may be any number of the cell isolation regions 142.

The number and configuration of the cell isolation region(s) 142 may be depend according to the size and properties of the cell(s) to be held. For example, the diameter or width of each cell isolation region 142 may correspond to the cell(s) to be held, such as the diameter of the cell.

If the cell holding device 140 includes a plurality of cell isolation regions 142, in some embodiments, the cell isolation regions 142 may have the substantially same configuration and/or size, different configurations/ and/or size, or a combination thereof. For example, the cell isolation regions 142 may be of a different configuration and/or size, for example, to hold different types of cells

If there are more than one cell isolation region 142, the regions 142 may be disposed or arranged in a pattern. As shown in FIGS. 1 through 3, 5 and 6, in some embodiments, the cell holding device 140 may include a plurality of cell isolation regions 142 in an array pattern.

However, the cell isolation region(s) 142 are not limited to that configuration and arrangement. The number and/or configuration of the cell isolation region(s) 142 may depend on the number, size, and properties of the cells(s) to be held. The cell holding device 140 may include any number of rows and columns of cell isolation regions 142. For example, the cell holding device 140 may include only one or one row of cell isolation regions 142. The cell holding device 140 may include a plurality of rows and columns of cell isolation regions 142. The cell isolation regions 142 may also be disposed anywhere along the length of the cell holding device 140. In some embodiments, the cell isolation regions may be disposed uniformly or evenly along the cell holding device 140. In other embodiments, the columns and/or rows of the cell isolation regions 142 may be staggered along the cell holding device 140. In further embodiments, the cell isolation regions 142 may be disposed unevenly, such as an offset from an edge of the cell holding device 140 or different amount of spacing between each cell isolation region 142 of the columns and/or rows.

The location and size of the cell holding device 140 may depend on the configuration (e.g., number and size) of the cell isolation region(s) 142. For example, the cell holding device 140 may have a width that substantially corresponds to the greatest width of the collection region 130, as shown in the figures. In some embodiments, as shown in FIG. 1, the cell holding device 140 may be disposed at or substantially near the middle section 135 of the collection region 130. In other embodiments, the cell holding device 140 may have a width that is smaller than the greatest width of the collection region 130.

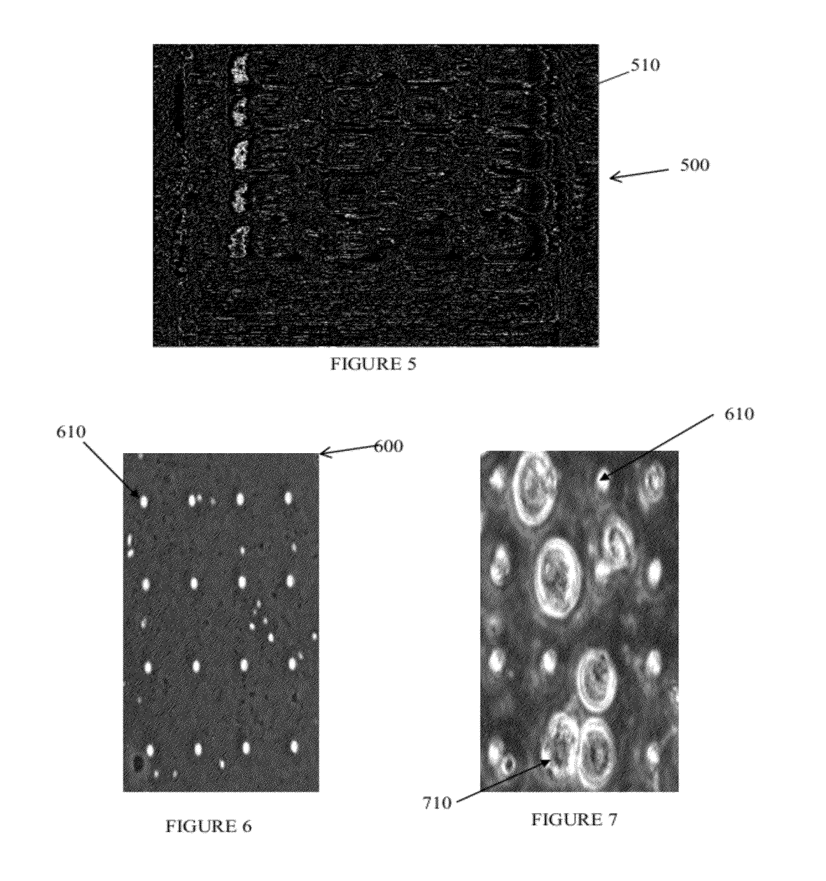

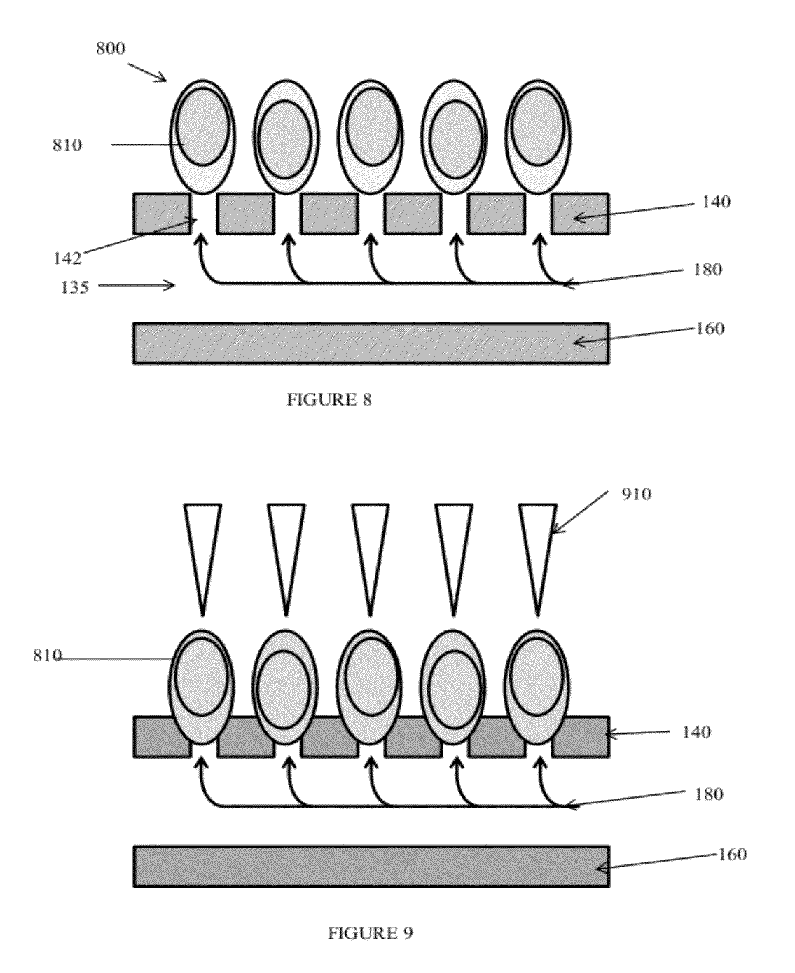

FIGS. 5 and 6 show examples of a cell holding device. FIG. 5 shows an example of a cell holding device 500. The cell holding device 500 may include a plurality of cell isolation regions 510 having a substantially square shape. FIG. 6 shows an example of a cell holding device 600. The cell holding device 600 may include a plurality of cell isolation regions 610 having a substantially circular shape. The cell isolation regions 610 are smaller than the cell isolation regions 510. FIG. 7 shows an example of cells 710 being held over the cell isolation regions 610.

In some embodiments, the cell isolation body 110 may include at least one fluid communication passage that is configured to be an outlet for the suspended fluid and/or an inlet to receive (and/or supply) positive or negative pressure. The fluid communication passage(s) may be an opening, a channel or fluid conduit of any length, depth, width and shape. The fluid communication passage(s) may include but is not limited to a microfluidic channel. In some embodiments, the cell isolation body 110 may include a passage may be configured to be an outlet for suspended fluid, an inlet for the suspend fluid and cells, an inlet to receive pressure, or a combination thereof. In other embodiments, the cell isolation body 110 may include more than one passage that is configured to be an outlet for suspended fluid, an inlet for the suspend fluid and cells, an inlet and an inlet to receive pressure, or a combination thereof. In some embodiments, the passage(s) may be in fluid communication with the region that includes the cell holding device 140. In some embodiments, the passage may include an opening disposed near or at a side of the cell isolation body 110.

As shown in FIG. 1, the cell isolation body 110 may include a passage 150 (also referred to as an outlet passage). The passage 150 may be in fluid communication with the collection region 130. In some embodiments, the passage 150 may communicate with the section 137. In other embodiments, the passage 150 may communicate with the additional or alternative sections of the region 130.

The passage 150 may be disposed any point or location on the cell isolation body 110, as well as at any depth. The passage 150 may be disposed between the collection region 130 and a side of the cell isolation body 110. In some embodiments, the passage body 150 may extend from the collection region 130 to a side of the cell isolation body 110. In some embodiments, the outlet passage 150 may extend along a portion of the length of the cell isolation body 110, as shown in FIG. 1. In other embodiments, the passage 150 may extend along a portion of the width of the cell isolation body 110. In some embodiments, the outlet passage 150 may include an outlet opening 152 disposed at a side of the cell isolation body 110. In some embodiments, the outlet opening 152 may be disposed at the side 114.

In some embodiments, the passage 150 may be disposed partially below or may have a side that is align with a bottom surface of at least one section of the collection region 130. In some embodiments, at least a portion of the passage 150 may communicate with a section disposed below the cell holding device 140 to be configured to be in communication with the fluid that passes through each cell isolation region and/or provide pressure. In some embodiments, the passage 150 may communicate with the sections 135 and 137.

In some embodiments, the passage 150 may have a width and depth that is configured to allow the suspension fluid or other fluid to flow through but not the cells to be separated. The passage 150 may also be configured to allow cells other than those to be separated to be flow through. The passage 150 may be a channel or fluid conduit of any length, depth, width and shape. The passage 150 may include but is not limited to a microfluidic channel. In some embodiments, the outlet passage 150 may have a width that is substantially smaller than the collection region 130. Also, the outlet passage 150 may have a and/or or be disposed at a depth greater than the depth of at least a portion of the collection region 130. In further embodiments, the outlet passage 150 may also have a tapered depth.

In some embodiments, the cell isolation body 110 may include a passage configured to supply positive or negative pressure. In some embodiments, the passage may be disposed below the cell holding device 140. In some embodiments, the passage may be the passage 150. In other embodiments, the passage may be different than the passage 150.

In some embodiments, the device 100 may further include a support substrate. The cell isolation body 110 may be disposed on or integrally formed with the support substrate 160. The support substrate 160 may be formed of any known materials and may include but is not limited to glass.

In further embodiments, the microfluidic device may include at least one sensor configured to determine states or flow characteristics of the regions of the microfluidic device or the passage of the cells and/or fluid through the microfluidic device.

In some embodiments, the cell isolation body 110 may be made from glass or plastic materials, such as hexanediol diacrylate (HDDA). In other embodiments, the cell isolation body 110 may be made of a different material.

In some embodiments, the device 100 may be a multi-layer structure. In some embodiments, the body 110 may include a plurality of layers. FIGS. 1 and 4 show an example of the multi-layer structure. In some embodiments, the body 110 may include at least three layers. In some embodiments, the body 110 may include a first layer, a second layer, and a third layer. The first layer may correspond to the top layer, the second layer may correspond to the middle layer, and the third layer may correspond to the bottom layer. In other embodiments, the body 110 may include additional layers.

In some embodiments, the first layer may include the cell receiving region 120, a passage 125, and at least one section of the collection region 130. In some embodiments, the first layer may include the section 133. The cell receiving region 120, the passage 125, and the section 133 may be disposed at the same depth. The cell receiving region 120, the passage 125, and the section 133 may also have the same depth. In some embodiments, the depth of the cell receiving region 120, the passage 125 and the section 133 may substantially correspond to the depth of the first layer. The bottom of the cell receiving region 120, the passage, and the section 133 may correspond to the top surface of the section layer.

The second layer may include the cell holding device 140. The cell isolating regions 142 may be disposed within the second layer. The cell holding device 140 may be disposed at the same depth as the cell receiving region 120, the passage 125, and the section 133. The cell holding device 140 may be disposed on the second layer so as to be substantially disposed within the section 135. The cell holding device 140 may be exposed and/or open communication with the cell receiving region 120.

The third layer may include at least one section of the region 130 and the passage 150. The third layer may include the sections 135 and 137. The sections 135 and 137, and the passage 150 may be disposed at the same depth. As shown in FIGS. 1 and 4, the sections 135 and 137, and the passage 150 may be disposed below the cell holding device 140. At least the section 137 and the passage 150 may also have the same depth. In some embodiments, the depth of the section 135 may correspond to the depth of the first and the second layer, and the depth of the section 137 and the passage 150 may correspond to the depth of the third layer. The bottom of the sections 135 and 137, and the passage 150 may correspond to the top surface of the support substrate 160.

In some embodiments, the microfluidic device may be fabricated using maskless micro-manufacturing techniques capable of creating multi-layer suspended polymer structures. In some embodiments, a digital micromirror device (DMD) may be used to polymerize hexanediol diacrylate (HDDA) with UV light. In further embodiments, conformal deposition of parylene may be applied to a cell holding device (example shown in FIG. 5) enabling excellent control of the hole size for a variety of different cell types.

The devices may be fabricated in one or more pieces that are then assembled. In one embodiment, separate layers of the devices may contain channels, regions, and/or holding devices. Layers of a device may be bonded together by clamps, adhesives, heat, anodic bonding, or reactions between surface groups (e.g., wafer bonding). Alternatively, the cell isolation body and/or the microfluidic devices may be fabricated as a single piece, e.g., using stereolithography or other three-dimensional fabrication techniques.

As shown in FIG. 4, the cell isolation body 110 may be disposed above the support substrate 160. The cell isolation body 110 may be formed of a polymerized HDDA. The regions may also be formed within the cell isolation body 110. After which, in some embodiments, the cell holding device 140 may be disposed within the cell isolation body 110, such as within the collection region 130. In some embodiments, the cell holding device 140 may be disposed above the passage 150. The cell holding device 110 may be formed of an unpolymerized HDDA.

However, fabrications of the microfluidic devices are not limited to these techniques. Variety of techniques may be employed to fabricate or manufacture devices of the disclosure, and the technique employed may be selected based in part on the material of choice. Materials include any rigid or flexible machinable material, such as glass, co-polymer, polymer or flexible elastomeric material. The materials may also include but are not limited to silicon, steel, nickel, poly(methylmethacrylate) (PMMA), polycarbonate, polystyrene, polyethylene, polyolefins, silicones (e.g., poly(dimethylsiloxane)), and combinations thereof. The materials may also be any known materials known in the art, such as those commonly used in microfluidic devices. The devices may also be formed according to any known methods in the art. Methods may include photolithography (e.g., stereolithography or x-ray photolithography), molding, embossing, silicon micromachining, wet or dry chemical etching, milling, diamond cutting, Lithographie Galvanoformung and Abformung (LIGA), electroplating, laser micromachining, thermoplastic injection molding, and compression molding.

The microfluidic devices according to embodiments may be part of a system. In some embodiments, the system may include at least one pressure source. The pressure source may be configured to employ positive and/or negative pressure. The pressure source may also be configured to control the flow rate. In some embodiments, the system may include a pressure source that employs both positive and negative pressure. In other embodiments, the system may include at least two pressure sources that each employs one kind of pressure. Examples of pressure source may include any known pressure source. The pressure source may include, but is not limited to a pump, such as a syringe pump, displacement pump, peristaltic pump, aspirator, and vacuum pump, and a pipette.

The microfluidic devices may be configured to isolate (or separate) and hold at least one cell by a pressure-driven flow. Positive pressure may be used to pump or cause the fluid to flow through the microfluidic devices 100, for example, from the receiving region 120 through the collection region 130. The positive pressure may be configured to cause the fluid to flow through the collection region 130 through the passage 150. The positive pressure may be configured to cause the fluid to flow through the region(s) of the microfluidic device. In some embodiments, the pressure source may be configured to cause the fluid to flow across the holding device 140 including the cell isolation region(s) 142 toward the outlet passage 150. While the fluid flows across the holding device 140, the cell(s) contained within the fluid may then be individually separated and held by each cell isolation region 142.

FIG. 8 shows an example of the microfluidic device 110 under controlled fluidic pressure. As shown in FIG. 8, cells 810 may be individually separated and held by each cell isolation region 142 when fluidic pressure 180 is applied.

The microfluidic devices may also be configured to release the held cells by a pressure-driven flow. In some embodiments, the cell isolation region(s) may be configured to release the cells when negative pressure is supplied. The microfluidic device may be configured to receive the release cells in any of the regions based on negative pressure. For example, the microfluidic device may be configured to receive the released cells in the cell receiving region 120 and/or the collection region 130.

In some embodiments, the system may further include at least one sensing device for measuring parameters of the cells. The sensing device may be configured to detect for a specific biological activity or a variety of biological activities. In further embodiments, the system may further include at least one delivery device for delivering materials to the cells. The sensing and delivery device may be any known device. The devices may be selected and adjusted based on the cells to be sensed/delivered as well as the parameters and materials to be sensed and delivered, respectively.

The delivery device may be configured to deliver any material to a cell. In some embodiments, the delivery device may configured to target a cell nucleus. The materials may be any known material, such as materials that may be used to determine biological activities of a cell or treat a cell, for example, gene therapy. The materials may include but are not limited to stem cells, gene therapeutics, drugs, antibodies, proteins, DNA, RNA, and nucleic acids. For example, engineered zinc finger nucleases may be delivered to the cells to specifically repair a genetic mutation.

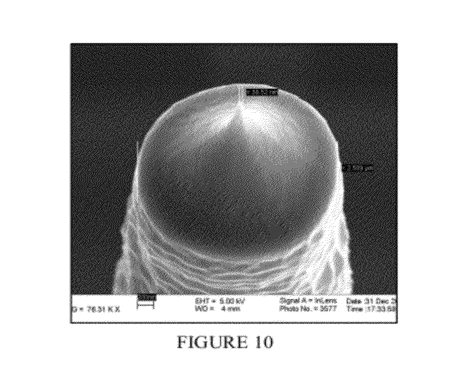

In some embodiments, the device may include a microneedle. In some embodiments, there may be a microneedle 910 for each separated cell 810, for example, as shown in FIG. 9. The microneedle may be of any size and may be adjusted according to the cells to be sensed or treated (by delivery of material). An example of a close-up of a microneedle is shown in FIG. 10. For example, in some embodiments, the microneedle may have a 40 nm tip, 28 μm tall and a 2.5 μm in diameter. In other embodiments, the microneedle may have a different size. The size of the microneedle may depend on the cell(s) to be separated. The microneedles may be configured to inject material at a calibrated distance.

In some embodiments, the system may further comprise a cell delivery device that delivers the cells to the microfluidic devices for separation. In some embodiments, the cell delivery device may be a pipette. In other embodiments, the cell delivery device is a robotic liquid handling system.

The system may further include a controller. The controller may be any known central processing unit, a processor, or a microprocessor. The controller may be coupled directly or indirectly to memory elements, such as random access memory (RAM), read only memory (ROM), disk drive, tape drive, etc., or a combinations thereof. The controller may be part of a computer system.

In some embodiments, the controller may be configured to control any component of the system, including but not limited to, the pressure source, the sensing device, as well as the delivery device. The controller may also be configured to control the microfluidic device, including the delivery of the cells for separation. The controller may also be configured to control the components of the system automatically, such as in a robotic liquid handling system.

In further embodiments, any steps of the process, including and not limited to delivering the cells to a microfluidic device, delivering the pressure to the microfluidic device, sensing the parameters of the cells, and delivering materials to the cells, may be automated.

While the disclosure has been described in detail with reference to exemplary embodiments, those skilled in the art will appreciate that various modifications and substitutions can be made thereto without departing from the spirit and scope of the disclosure as set forth in the appended claims. For example, elements and/or features of different exemplary embodiments may be combined with each other and/or substituted for each other within the scope of this disclosure and appended claims.

1. A device configured to separate, hold and release at least one cell suspended in a fluid, comprising:
a region configured to receive the fluid including at least one cell;
a cell holding device that is in open communication with the region and includes at least one cell isolation region configured to releasably separate and hold each cell; and
a passage that is configured to receive and supply pressure to the cell holding device.

2. The device according to claim 1, wherein the cell isolation region includes an opening that is configured to support a portion of a surface of each cell.

3. The device according to claim 1, wherein the region is disposed substantially at a same depth as the cell holding device.

4. The device according to claim 1, further comprising:
a second region that includes the cell holding device and is in fluid communication with the region, the second region including a plurality of sections.

5. The device according to claim 4, wherein the region is disposed above at least one section of the second region.

6. The device according to claim 1, wherein the cell holding device is disposed above the passage.

7. The device according to claim 1, wherein the cell holding device includes an array of a plurality of cell isolation regions.

8. The device according to claim 7, wherein each of the plurality of cell isolation regions is of a substantially a same size.

9. The device according to claim 7, wherein the plurality of cell isolation regions are of different sizes.

10. The device according to claim 1, further comprising:
a support substrate, wherein the region, cell holding device and passage are disposed above the support substrate.

11. A device comprising:
a cell receiving region configured to receive the fluid including at least one cell;
a collection region that is in fluid communication with the cell receiving region, the collection region including a cell holding device configured to releasably separate and hold at least one cell; and
a passage disposed adjacent to the collection region,
wherein the passage is disposed below the cell receiving region and the cell holding device.

12. The device according to claim 11, wherein the collection region includes at least a first section, a second section, and a third section, the first section and the third section having a tapered width.

13. The device according to claim 12, wherein the cell holding device is disposed within the second section of the collection region.

14. The device according to claim 11, wherein the passage extends from the collection region to a side of the device.

15. The device according to claim 14, wherein the passage is configured to allow the fluid to exit the collection region.

16. The device according to claim 14, wherein the passage is configured to prevent the at least one cell to be separated to exit the collection region.

17. The device according to claim 14, wherein the passage is configured to receive and supply pressure.

18. A system that is configured to controllably separate, hold and release at least one cell suspended in a fluid, comprising:
a microfluidic device, comprising:
a region configured to receive the fluid including at least one cell;
a cell holding device that is in open communication with the region and is configured to releasably and individually separate and hold each cell; and
a passage extending from the region to a side of the cell holding device, the passage being configured to control movement of the at least one cell within the microfluidic device.

19. The system according to claim 18, further comprising:
a pressure source configured to connect to the passage to supply at least one of a positive or negative pressure.

20. The system according to claim 18, further comprising:
at least one delivery device configured to deliver a material to a separated cell.
